(19) United States
(12) Patent Application Publication
(10) Pub. No.: US 2021/0304849 A1
(43) Pub. Date: Sep. 30, 2021

(54) BIOCHEMICAL DATA PROTEIN ACTING ON BIONIC DATA CELL BODY

(71) Applicant: Xiaojun Zhu, Hangzhou, Zhejiang (CN)

(72) Inventors: Xiaojun Zhu, Hangzhou, Zhejiang (CN); Lie Xiao, Hangzhou, Zhejiang (CN)

(21) Appl. No.: 17/267,042

(22) PCT Filed: Aug. 16, 2018
(86) PCT No.: PCT/CN2018/100857

§ 371 Date: Feb. 9, 2021

(51) Int. Cl. G16B 50/30; G16B 50/10; G16B 40/10; G16B 30/00

(52) U.S. Cl. CPC G16B 50/30; G16B 30/00; G16B 40/10; G16B 50/10

(57) ABSTRACT

Provided is a biochemical data protein acting on a biomimetic data cell body. The data cell body includes a data ontology and a data chromosome; the data ontology is data to be transmitted; the data chromosome is attribute information of the data ontology. The biochemical data protein is used for matching a data cell body, or updating the data cell body, or performing an operation on the data chromosome of the data cell body. The biochemical data protein includes: an initiator, an action, a carrier, and a recipient, wherein the initiator is the initiator of the biochemical data protein, the action is command content needing to be completed by the biochemical data protein, the carrier is the data ontology, and the recipient is an action object or an output object of the action.

### FIELD

The invention relates to the Internet field, and more specifically, to a biochemical data protein acting on a bionic data cell body.

### BACKGROUND

The Internet has become a way of life for people. People communicate, shop, send files, release information, etc., through the Internet. Users will continuously generate data and send these data through the Internet. However, the existing Internet does not consider the user's control over the data in the process of data transmission. Once the data is disseminated through the Internet, the user loses the control over the data, which brings great hidden dangers to the user's data security.

Existing Internet data is still pure file description data, such as text, video, picture, audio, etc. The file format is only a representation of its content. Although different encoding methods can be used, the data itself does not have corresponding attribute information. All operations need to be completed by the outside world.

### SUMMARY

The technical problem to be solved by the invention is to provide a biochemical data protein acting on a bionic data cell body in response to the above-mentioned defects in the prior art.

The technical solution adopted by the invention to solve its technical problems is to construct a biochemical data protein acting on a bionic data cell body, the data cell body comprises a data ontology and a data chromosome, and the data ontology is data to be transmitted; the data chromosome is the attribute information of the data ontology;

the biochemical data protein is configured to match the data cell body, or update the data cell body, or perform operations on the data chromosome of the data cell body;

the biochemical data protein comprises: an initiator, an action, a carried object, and a recipient, wherein the initiator is the initiator of the biochemical data protein, the action is a command content to be completed by the biochemical data protein, and the carried object is the data chromosome, and the recipient is an acting object or output object of the action.

Further, the biochemical data protein acting on a bionic data cell body of the invention comprises: an ordinary biochemical data protein, a historical biochemical data protein, and an operational biochemical data protein, wherein,

the ordinary biochemical data protein is configured for query and verification;

the historical biochemical data protein is configured to change the data ontology or the data chromosome; and

the operational biochemical data protein is configured to perform operations on the data cell body.

In some embodiments, according to the biochemical data protein acting on a bionic data cell body of the invention, the data cell body has a unique identifier ID, and the unique identifier ID is a unique identification code of a user; or the data cell body is identified by content and uses Data URI schema format in the http protocol;

the historical biochemical data protein has a unique identifier ID, and the unique identifier ID is a unique identification code of the user.

Further, according to the biochemical data protein acting on the bionic data cell body of the invention, the action comprises:

a Send command for sending or forwarding data;

an Update command for updating data;

a Delete command for deleting data;

a Withdrawal command for withdrawing data;

a Statistics command for statistics; and

a Make command for making the data ontology and the data chromosome.

Further, the biochemical data protein acting on a bionic data cell body according to the invention further comprises:

a Regist command for carrying out registration with a long link and indicating the identity of a link creator;

a Query command for querying historical data, and tracing and triggering the receipt of offline messages;

a State command for indicating whether the reception state is normal, wherein the state command comprises a state command of successful reception (success) and a state command of failed reception (fail);

a Notify command for notifying changes in the data chromosome of the data cell body;

a Trace command for tracing data;

a Compute command for computing the data cell body; and

a Result command for returning query or computation results.

Further, according to the biochemical data protein acting on a bionic data cell body of the invention, all commands in the action each include an optional attribute for asynchronous systems. If a sender carries the optional attribute, a returner needs to carry the optional attribute.

Further, according to the biochemical data protein acting on a bionic data cell body of the invention, the data structures of the initiator, the action, the carried object, and the recipient are all in a form of ID plus attribute table;

the ID is the content of the data cell body; if there is no ID or there are multiple IDs, preset characters are used instead;

if one of the initiator, the action, the carried object, and the recipient does not have an attribute table, an ID string is retained.

Further, according to the biochemical data protein acting on a bionic data cell body of the invention, the data chromosome comprises:

an identity gene for indicating identity information of the data cell body;

an internal characteristic gene for indicating an internal data state of the data cell body;

a vital characteristic gene for describing vital characteristics of the data cell body;

a state characteristic gene for describing the current state of the data cell body; and

a system control gene for system control.

Further, according to the biochemical data protein acting on a bionic data cell body of the invention, the identity gene comprises: the unique identifier ID of the data cell body and MD5 digest information;

the internal characteristic gene comprises: data type, MD5 digest value, character size value, text encoding format information, file extension, and creation time;

the vital characteristic gene comprises: authority information for limiting the viewing authority of the data cell body, spread width information for limiting the viewing range of the data cell body, spread depth information for limiting the number of forwarding layers of the data cell body, life information of the data cell body, and ownership information for limiting the ownership of data; the authority information comprises an authority defines that data can be seen by everyone and searchable, an authority defines that data can be seen by everyone but not searchable, an authority defines that data can be seen by a creator of the data cell body and preset users and searchable, and an authority defines that data can be seen by the creator of the data cell body and preset users but not searchable;

the state characteristic gene comprises: the current spread width information of the data cell body and the current spread depth information of the data cell body;

the system control gene comprises the sequence number of a asynchronous protocol command frame configured to matching the command result and an activation gateway configured to verify the identification.

Further, according to the biochemical data protein acting on a bionic data cell body of the invention, the data chromosome is composed of key-value pairs, wherein the key is a character string;

the data ontology comprises one or more of a text, a video, a picture, an audio, a file, a name card, a pdf document, an office document, and an xml document.

A biochemical data protein acting on a bionic data cell body according to the invention has the following beneficial effects: the data cell body comprises a data ontology and a data chromosome; the data ontology is data to be transmitted; the data chromosome is the attribute information of the data ontology; the biochemical data protein is configured to match the data cell body, or update the data cell body, or perform operations on the data chromosome of the data cell body; the biochemical data protein comprises: an initiator, an action, a carried object, and a recipient, wherein the initiator is the initiator of the biochemical data protein, the action is a command content to be completed by the biochemical data protein, and the carried object is the data ontology, and the recipient is an acting object or output object of the action. By implementing the invention, provided are a data cell body similar to a biological cell, and a biochemical data protein for matching the data cell body, or updating the data cell body, or performing operations on the data chromosome of the data cell body, and the biochemical data protein is configured to perform control over the data cell body.

### BRIEF DESCRIPTION OF THE DRAWINGS

The invention will be further described with reference to the accompanying drawings and specific embodiments. In the drawings,

FIG. 1 is a schematic structural diagram of the data cell body of the invention;

FIG. 2 is a schematic structural diagram of the data cell body and the data protein according to Embodiment 1 of the invention;

FIG. 3 is a schematic structural diagram of the data cell body and the data protein according to Embodiment 2 of the invention.

### DESCRIPTION OF THE EMBODIMENTS

In order to have a clearer understanding of the technical features, objectives and effects of the invention, the specific embodiments of the invention will now be described in detail with reference to the accompanying drawings.

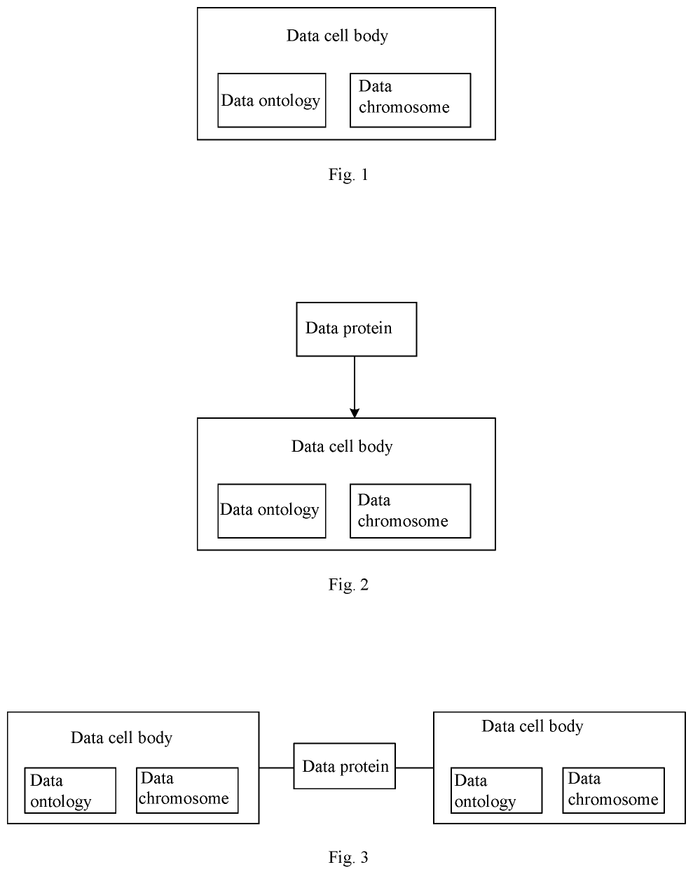

Referring to FIG. 1, the data cell body of the invention comprises a data ontology and a data chromosome, and the data ontology is data to be transmitted. The data cell body is generated by a terminal. After the terminal generates the data cell body, a control over the data cell body is obtained by the terminal and realized through the data chromosome. The user is the owner and user of the terminal, and the data generated by the terminal is also the data made under the control of the user, thus realizing the user's control over the data. In some embodiments, the data ontology includes but is not limited to a text, a video, a picture, an audio, a file, a name card, a pdf document, an office document, and an xml document, and data formats used in the existing Internet and terminals, and new ones generated in the future can all be used as the data ontology, which is not limited by the invention. In addition, the encoding method of the data ontology can be selected according to needs, so as to be suitable for terminal processing or transmission on the Internet.

The data chromosome is the attribute information of the data ontology, and the data chromosome determines the appearance, shape, life cycle, visible range, and other attributes of the data cell body. The data chromosome is also a feature entry for the outside world to find the data cell body, and the data chromosome is composed of key-value pairs, wherein the key is a character string. The data chromosome can be copied, modified, converted, spread, etc. The change of the data chromosome does not mean the change of the data ontology. Suppose that a video cell, i.e., a video, is considered as the data ontology and the number of people who have watched the video is considered as the data chromosome of the video cell, as the number of people who have watched the video increases, the corresponding data chromosome has been changing, but the video itself has not changed.

It should be noted that the object of the attribute information referred to by the data chromosome here is the data ontology, that is, the attribute information of the data ontology which is considered as a whole, and the attribute information is not related to the specific content of the data ontology. The data ontology may also be understood as an encapsulated black box in which the data contained can be diverse. In addition, the attribute information referred to by the data chromosome here is different from those used to describe a file. For example, a picture is a file. The attributes of the picture include size, creation date, and resolution etc., and all these attributes are used to describe the characteristics of the picture itself. In the invention, the attribute information referred to by the data chromosome is considering the related data of the picture as a whole, and then describing the attribute features such as appearance, shape, life cycle, and visible range of the whole.

In some embodiments, the data chromosome comprises: an identity gene, an internal characteristic gene, a vital characteristic gene, a state characteristic gene, and a system control gene, wherein,

the identity gene is configured to indicate identity information of the data cell body. The identity gene comprises: a unique identifier ID of the data cell body and MD5 digest information. In the process of program implementation, the type of unique identifier ID may be a string class, and the MD5 digest information may be of a string class. The generation of the MD5 digest information can refer to the prior art.

The internal characteristic gene is configured to indicate the internal data state of the data cell body. The internal characteristic gene comprises: data type, MD5 digest value, character size value, text encoding format information, filename extension, and creation time. The data type here can be text/plan, image/jpg, mp4/video, etc., and is similar to the content-type in the http protocol. In the process of program implementation, the data type can be of a string class, the MD5 digest value can be of a string class, the character size value can be of an integer class, the text encoding format information can be a string class, the filename extension can be of a string class, and the creation time can be of a datatime class.

A vital characteristic gene is configured to describe vital characteristics of the data cell body. The vital characteristic gene comprises: authority information for limiting the viewing authority of the data cell body, spread width information for limiting the viewing range of the data cell body, spread depth information for limiting the number of forwarding layers of the data cell body, life information of the data cell body, and ownership information for limiting the ownership of data.

Further, the authority information comprises an authority defines that data can be seen by everyone and searchable, an authority defines that data can be seen by everyone but not searchable, an authority defines that data can be seen by the creator of the data cell body and preset users and searchable, and an authority defines that data can be seen by the creator of the data cell body and the preset users but not searchable. In the process of program implementation, the authority information can use enumeration classes. For example, the values can be defined as public, private, and protected respectively. The authority “public” means data can be seen by everyone and searchable; the authority “private” means that data can be seen by the owner (data creator) and related people but not searchable; the authority “protected” means data can be seen by everyone but not searchable. With this setting, it is limited whether the data can be viewed and searched, so as to effectively control the data.

In the process of program implementation, the spread width information can be of an integer class to limit the viewing range of the data cell body, that is, to limit the number of people allowed to see the data cell body. With this setting, at the beginning of the user creating the data, the user can limit the scope of the data cell body to be viewed by setting the spread width information, so as to effectively control the data. The spread depth information can be of an integer class. The spread depth here refers to the number of layers at which the data cell body is allowed to be forwarded, and one forwarding can be considered as one layer.

The life information of the data cell body is set when the data cell body is created. After the life information is set, the timing starts when the data cell body is created. When the timing reaches a preset time, the data cell body is stopped from being transmitted and viewed. In some embodiments, the data cell body can also be automatically destroyed.

The ownership information can be written when the data cell body is created. For example, a terminal ID, a user name, a mobile phone number, etc. are used as the user's authority representative information and thus function as the ownership information, so that the ownership and control of the data cell body can be determined based on the ownership information.

The state characteristic gene is configured to describe the current state of the data cell body. The state characteristic gene comprises: the current spread width information of the data cell body and the current spread depth information of the data cell body. As the state description information, the state characteristic gene will change with the state of the data cell body. In the process of program implementation, the current spread width information can be of an integer class, and the current spread depth information can of an integer class.

The system control gene is configured for system control. The system control gene comprises the sequence number of an asynchronous protocol command frame configured to matching the command result and an activation gateway configured to verify the identification. In the process of program implementation, the sequence number of the asynchronous protocol command frame can be of an integer class, and the activation gateway can be of an integer class.

In summary, the data cell body of the invention not only comprises the data to be transmitted, and further comprises the attribute information of these data. The attribute information makes the data vital, and thus more bionic operations can be done.

FIGS. 2 and 3, the invention also provides data protein acting on a bionic data cell body. The data protein is configured to perform a preset function on the data cell body or between data cell bodies. The data protein comprises operation information corresponding to the preset function, and the preset function can be completed by analyzing the data protein. The data cell body comprises a data ontology and a data chromosome. The data ontology is data to be transmitted, and the data chromosome is the attribute information of the data ontology. The specific definition of the data cell body refers to the above description.

The data protein can operate the data cell body in the following three ways:

The first way: the data cell body is operated by receiving data proteins from the outside world.

The second way: the data cell body activates the data protein at a preset time point and thus is operated.

The third way: the data protein operates the data cell body spontaneously according to its internal settings.

Specifically, the data protein comprises: a characteristic data protein, a matching data protein, a defense data protein, and a biochemical data protein, wherein,

The characteristic data protein is configured to classify and characterize the data cell body. The characteristic data protein comprises an array composed of a key value and tags, for example, an array of the “key” being “tag”.

The matching data protein is configured to search and query the data cell body. The matching data protein searches and queries the internal characteristic gene in the chromosome of the data cell body. The matching protein is a full-text index. If the authority information in the vital characteristic gene of the data chromosome of the data cell body is set to public, and because the authority “public” defines that data can be seen by everyone, the data cell body will have a full-text index. The matching protein is configured to search and query the cell. When the cell is made, the corresponding data chromosome is also made. If the vital characteristic gene matches, the full-text index will be established at the same time.

The defense data protein is configured for key matching with the data cell body. The defense data protein is matched with the secret key of the data cell body, and the data cell body is opened if the match is successful. The defense protein is the keyhole to open the data cell body. When the defense protein meets a protein (secret key) matching the defense protein, the data cell body can be opened. In an organic system, all data cell bodies are encrypted, and keys for decryption are proteins that can be matched by key proteins on cell membranes.

Further, the data cell body of the invention comprises a tissue data cell for combining a plurality of data cell bodies into a whole, and the plurality of data cells perform activities according to the organization rules in the tissue data cell. The tissue is a concept of tissue borrowed from physiology, which refers to a whole body composed of data cell bodies with a specific function and carrying out organized and regular activities according to predetermined rules. A complete event of a tissue is an event itself. A tissue event comprises multiple sub-events. There are different types of tissue activities, and each specific type can have multiple plans. The tissue activities are performed in an orderly manner in accordance with the plans. The plans themselves are also data cell bodies, specifically stem cells. For example, the types of tissue activities include meetings, elections, and workflows. By defining the namespace of the system category, the action of the tissue itself can be defined. For an action belonging to the tissue, a namespace should be added before the action, separated by a colon therebetween, such as jbbm:start, jbpm:signal, and jbpm:end. A plan is defined according to a tissue category. For example, the tissue category is workflow, the plan is a workflow definition file, such as a process definition document (an XML file) of bpmn or jbpm.

The biochemical data protein is configured to match the data cell body or update the data chromosome of the data cell body. The biochemical data protein comprises an operational protein configured to perform operations on the data chromosome of the data cell body.

Further, the biochemical data protein is configured to match the data cell body, or update the data cell body, or perform operations on the data chromosome of the data cell body; the biochemical data protein comprises: an initiator, an action, a carried object, and a recipient, wherein the initiator is the initiator of the biochemical data protein, the action is a command content to be completed by the biochemical data protein, and the carried object is the data chromosome, and the recipient is an acting object or output object of the action.

According to the different tissue functions, biochemical data proteins are divided into: an ordinary biochemical data protein, a historical biochemical data protein, and an operational biochemical data protein, wherein

The ordinary biochemical data protein is configured for query and verification, and will not change the data in the data ontology of the data cell, nor will it change the attribute information in the data chromosome.

The historical biochemical data protein is configured to change the data ontology or data chromosome. The historical biochemical data protein comprises some instructions that can change the data ontology or data chromosome. Once such instructions are executed, the data ontology or data chromosome will be changed. Certainly, the data ontology and the data chromosome may also be changed at the same time.

The operational biochemical data protein is configured to perform operations on the data cell body. Operational biochemical data protein is a command configured to perform operations on events or the data cell body. The command can be an ordinary biochemical data protein or a historical biochemical data protein, depending on whether the result of the operations will change the composition of the data cell body. If the result of the operations causes the addition of a new data cell body or changes in the data chromosome, that is, changing the attribute information of the data ontology, this command is a historical biochemical data protein, otherwise it is an ordinary biochemical data protein.

The data cell body in the invention has a unique identifier ID, the unique identification ID is a unique identification code of the user, and the unique identifier ID can be generated by a client terminal or a server. Alternatively, the data cell body can also be identified by content and uses the Data URI schema format in the http protocol, and the Data URI schema format can refer to the prior art.

Similarly, the historical biochemical data protein has a unique identifier ID, and the unique identifier ID is a unique identification code of the user. The unique identification ID can be generated by the client terminal or the server.

Further, according to the biochemical data protein acting on the bionic data cell body of the invention, the action comprises:

a Send command for sending or forwarding data;

an Update command for updating data;

a Delete command for deleting data;

a Withdrawal command for withdrawing data; Withdrawal refers to the withdrawal of all nodes downstream of a node on a data spread chain;

a Statistics command for carrying out statistics; and

a Make secret for making the data ontology and the data chromosome.

a Regist command for carrying out registration with a long link and indicating the identity of a link creator;

a Trace command for querying historical data, tracing and triggering the receipt of offline messages;

a State command for indicating whether the reception state is normal, wherein the state command comprises the state command of successful reception (success) and the state command of failed reception (fail);

a Notify command for notifying changes in the data chromosome of the data cell body;

a Trace command for tracing data;

a Compute command (agg) for computing the data cell body;

a Result command for returning query or computation results.

It can be understood that one or more of the various commands of the above actions can be selected and implemented according to requirements. Therefore, selecting one or more commands for implementation belongs to the protection scope of the invention.

Further, all commands in an action include an optional attribute seq for asynchronous systems, which is used for corresponding to the commands of the asynchronous systems. If the sender carries the optional attribute seq, the returner needs to carry the optional attribute seq. For example, the optional attribute seq is an unsigned integer with a step size of 1 in the same session. A request with the optional attribute seq is an asynchronous request, and the one without the optional attribute seq is a synchronous request.

In some embodiments, the data structures of the initiator, the action, the carried object, and the recipient of the biochemical data protein are all in a form of ID+attribute table; ID is the content of the data cell body; if there is no ID or there are multiple IDs, the preset characters are used instead. If one of the initiator, the action, the carried object, and the recipient does not have an attribute table, an ID string is retained.

By implementing the invention, provided are a data cell body similar to a biological cell, and a biochemical data protein for matching the data cell body, or updating the data cell body, or performing operations on the data chromosome of the data cell body, and the biochemical data protein is configured to complete the control over the data cell body.

The above embodiments are only to illustrate the technical concept and features of the invention, and their purpose is to enable those familiar with the art to understand the content of the invention and implement the invention accordingly, and cannot limit the protection scope of the invention. All equivalent changes and modifications made in accordance with the scope of the claims of the invention shall fall within the scope of the claims of the invention.

What is claimed is:

1. A biochemical data protein acting on a bionic data cell body, wherein the data cell body comprises a data ontology and a data chromosome, and the data ontology is data to be transmitted; the data chromosome is attribute information of the data ontology;
the biochemical data protein is configured to match the data cell body, or update the data cell body, or perform operations on the data chromosome of the data cell body; and
the biochemical data protein comprises: an initiator, an action, a carried object, and a recipient, wherein the initiator is the initiator of the biochemical data protein, the action is a command content to be completed by the biochemical data protein, the carried object is the data chromosome, and the recipient is an acting object or output object of the action.

2. The biochemical data protein acting on a bionic data cell body according to claim 1, wherein the biochemical data protein comprises an ordinary biochemical data protein, a historical biochemical data protein, and an operational biochemical data protein, wherein,
the ordinary biochemical data protein is configured for query and verification;
the historical biochemical data protein is configured to change the data ontology or the data chromosome; and
the operational biochemical data protein is configured to perform operations on the data cell body.

3. The biochemical data protein acting on a bionic data cell body according to claim 2, wherein the data cell body has a unique identifier ID, and the unique identifier ID is a unique identification code of a user; or the data cell body is identified by content and uses a Data URI schema format in the http protocol;
the historical biochemical data protein has a unique identifier ID, and the unique identifier ID is a unique identification code of the user.

4. The biochemical data protein acting on a bionic data cell body according to claim 1, wherein the action comprises:
a Send command for sending or forwarding data;
an Update command for updating data;
a Delete command for deleting data;
a Withdrawal command for withdrawing data;
a Statistics command for carrying out statistics; and
a Make command for making the data ontology and the data chromosome.

5. The biochemical data protein acting on a bionic data cell body according to claim 4, wherein the action further comprises:
a Regist command for carrying out registration with a long link and indicating the identity of a link creator;
a Query command for querying historical data, and tracing and triggering the receipt of offline messages;
a State command for indicating whether the reception state is normal, wherein the state command comprises a state command of successful reception and a state command of failed reception;
a Notify command for notifying changes in the data chromosome of the data cell body;
a Trace command for tracing data;
a Compute command for computing the data cell body; and
a Result command for returning query or computation results.

6. The biochemical data protein acting on a bionic data cell body according to claim 1, wherein all commands in the action each comprise an optional attribute for asynchronous systems; if a sender carries the optional attribute, a returner needs to carry the optional attribute.

7. The biochemical data protein acting on a bionic data cell body according to claim 1, wherein the data structures of the initiator, the action, the carried object, and the recipient are all in a form of ID plus attribute table;
the ID is the content of the data cell body; if there is no ID or there are multiple IDs, preset characters are used instead;
if one of the initiator, the action, the carried object, and the recipient does not have an attribute table, an ID string is retained.

8. The biochemical data protein acting on a bionic data cell body according to claim 1, wherein the data chromosome comprises:
an identity gene for indicating identity information of the data cell body;
an internal characteristic gene for indicating an internal data state of the data cell body;
a vital characteristic gene for describing vital characteristics of the data cell body;
a state characteristic gene for describing the current state of the data cell body; and
a system control gene for system control.

9. The biochemical data protein acting on a bionic data cell body according to claim 8, wherein the identity gene comprises: a unique identifier ID of the data cell body and MD5 digest information;
the internal characteristic gene comprises: data type, MD5 digest value, character size value, text encoding format information, filename extension, and creation time;
the vital characteristic gene comprises: authority information for limiting the viewing authority of the data cell body, spread width information for limiting the viewing range of the data cell body, spread depth information for limiting the number of forwarding layers of the data cell body, life information of the data cell body, and ownership information for limiting the ownership of data; the authority information comprises an authority defines that data can be seen by everyone and searchable, an authority defines that data can be seen by everyone but not searchable, an authority defines that data can be seen by a creator of the data cell body and preset users and searchable, and an authority defines that data can be seen by the creator of the data cell body and preset users but not searchable;
the state characteristic gene comprises: the current spread width information of the data cell body and the current spread depth information of the data cell body;
the system control gene comprises a sequence number of an asynchronous protocol command frame configured to matching the command result and an activation gateway configured to verify the identification.

10. The biochemical data protein acting on a bionic data cell body according to claim 1, wherein the data chromosome is composed of key-value pairs, wherein the key is a character string;
the data ontology comprises one or more of text, video, picture, audio, file, name card, pdf document, office document, and xml document.
